(19) United States
(12) Patent Application Publication
(10) Pub. No.: US 2004/0049160 A1
(43) Pub. Date: Mar. 11, 2004

(54) Safety hypodermic syringe

(76) Inventors: Chung-Chieh Hsieh, Taichung City (TW); Ming-Kan Hung, Taichung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/237,953

(22) Filed: Sep. 10, 2002

(51) Int. Cl. A61M 5/32

(52) U.S. Cl. 604/195; 128/919

(57) ABSTRACT

A safety hypodermic syringe includes a barrel and a base is movably received in the barrel. A cylindrical connection piece is engaged with the engaging recess. A needle cannula unit is fixed connected to the base and extends out from the barrel. A plunger has an insertion which is inserted through the hole in the connection piece and into the passage in the base. The insertion is securely engaged with the connection piece. A breaking groove is defined in an outer periphery of the plunger and located close to the second end of the plunger. The base together with the needle cannula unit are pulled in the barrel after injection and the plunger is broken to leave the needle cannula unit in the barrel.

### FIELD OF THE INVENTION

The present invention relates to a safety hypodermic syringe wherein the plunger is pulled back and broken to allow the needle cannula and the locking tip to be left in the barrel after the injection is completed.

### BACKGROUND OF THE INVENTION

A conventional hypodermic syringe for injecting medicine into patient's body generally includes a barrel with a plunger movably inserted therein from a first end thereof and a locking tip with a needle cannula is fixed to a second end of the barrel. Medicine is sucked in the space of the barrel and the plunger has a stopper which seals the inner periphery of the barrel so as to push the medicine out from the needle cannula. A cap is used to re-mount the needle cannula so as to prevent the needle cannula from hurting people. It is dangerous for people being hurt by the needle cannula which is attached with medicine, the patient's blood or body liquid. Nevertheless, the re-cap action is proceeded in a hospital hundreds of time in one day and the nurses work under a potential risk of being hurt by the discarded needle cannulas.

The present invention intends to provide a safety hypodermic syringe, the needle cannula can be pulled back into the barrel and left in the barrel after the injection action.

### SUMMARY OF THE INVENTION

In accordance with one aspect of the present invention, there is provided a safety hypodermic syringe which comprises a barrel having an opening defined in a first end thereof through which the plunger is inserted. A base and a connection piece are movably received in the barrel and a passage is defined through the base. A needle cannula unit is fixed connected to the base and extends out from the second end of the barrel.

The plunger has an insertion which is inserted through the connection piece and into the passage in the base. The insertion is securely engaged with the connection piece. A stopper is mounted to a root portion of the insertion. A breaking groove is defined in an outer periphery of the plunger and located close to the second end of the plunger.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a cross sectional view to show the safety hypodermic syringe of the present invention;

FIG. 2 is an exploded view to show the safety hypodermic syringe of the present invention;

FIGS. 3A and 3B show the plunger is inserted into the barrel to be engaged with the base;

FIG. 3C shows that the plunger has engaged with the base and to be pulled back;

FIG. 3D shows the plunger is pulled back after injection and broken at the breaking groove;

FIG. 4 is an enlarged view to show the engagement of the insertion of the plunger and the base; and

FIG. 5 is an enlarged view to show that the flange of the connection piece is deformed and disengaged from the first groove in the barrel.

### DETAILED DESCRIPTION OF THE PREFERRED EMBODIMENT

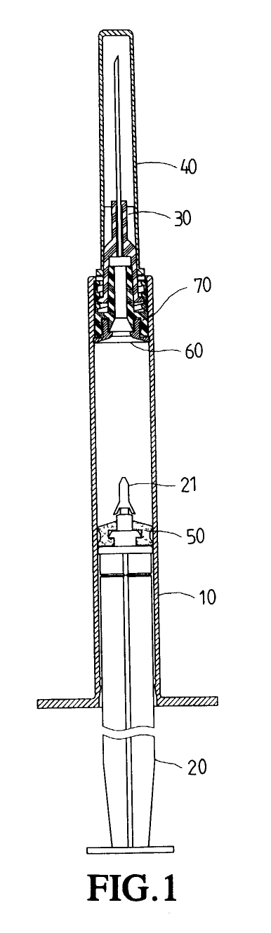

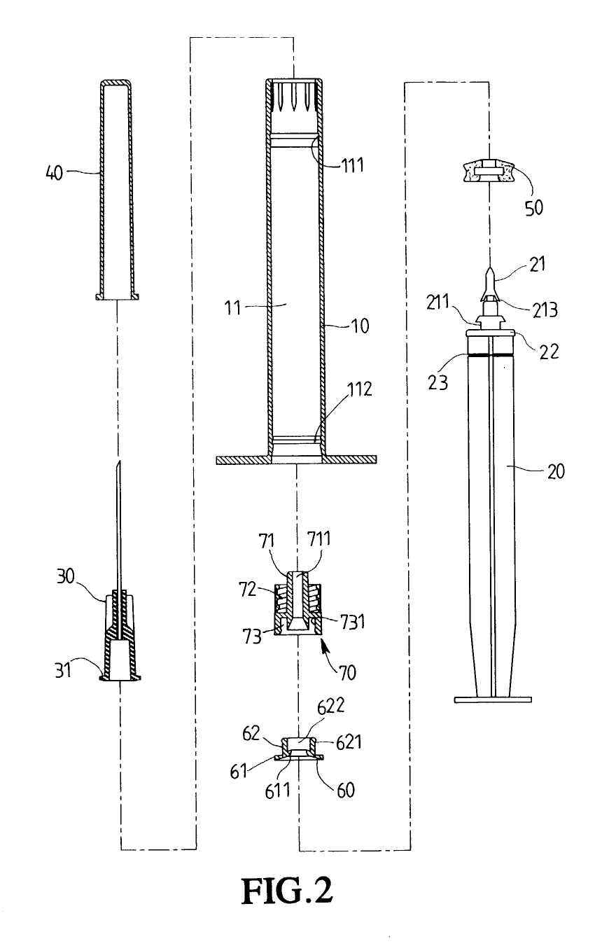

Referring to FIGS. 1, 2 3A and 3B, the safety hypodermic syringe of the present invention comprises a barrel 10 having a space 11 defined therein and an opening is defined in a first end of the barrel 10. The opening communicates with the space 11. The barrel 10 has a first groove 111 defined in an inner periphery thereof and is located close to the second end of the barrel 10. A second groove 112 is defined in the inner periphery of the barrel 10 and located close to the first end of the barrel 10.

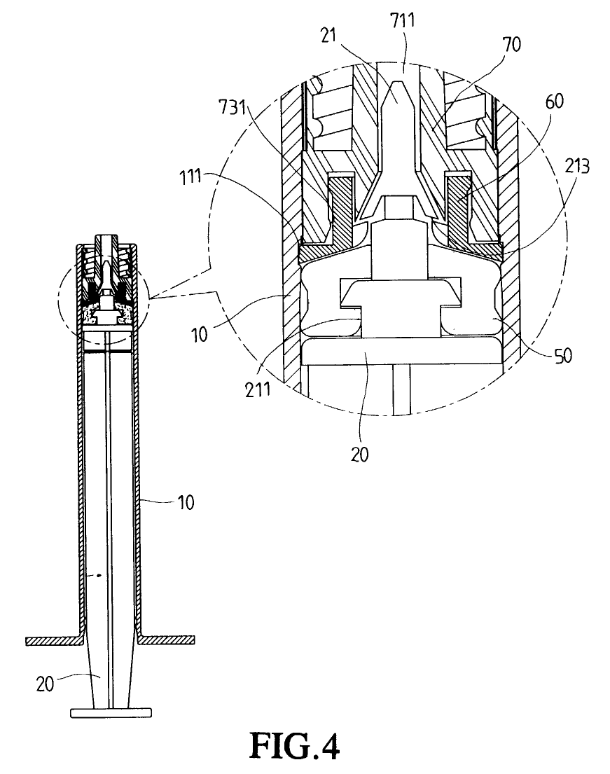

A base 70 is movably received in the barrel 10 and includes a tube 71 located in the base 70, a passage 711 defined through the tube 71. An engaging recess 73 is defined in a first end of the base 70 and a threaded inner periphery 72 is defined in a second end of the base 70. A cylindrical connection piece 60 has a hole 622 defined therethrough and is engaged with the engaging recess 73. The connection piece 60 has a flange 61 on an outer periphery thereof and a ridge 611 extends from an inner periphery of the hole 622 of the connection piece 60. The base 70 has a first connection protrusion 731 extending from an inner periphery of the engaging recess 73 and the connection piece 60 has a second connection protrusion 621 on an outer periphery thereof. The first connection protrusion 731 is engaged with the second connection protrusion 621. The flange 61 of the connection piece 60 is flexible and engaged with the first groove 111 in the barrel 10 as shown in FIG. 4.

A needle cannula unit 30 has a flange 31 which is engaged with the threaded inner periphery 72 of the base 70 and the needle cannula extends out from the second end of the barrel 10. A cap 40 can be mounted on the needle cannula unit 30.

A plunger 20 has a thumb rest on a first end thereof and an insertion 21 extends from a second end of the plunger 20. A groove 211 is defined in the root portion of the insertion 21 and the stopper 50 is engaged with the groove 211 in the root portion of the insertion 21. An enlarged skirt 213 extends from an outer periphery of the insertion 21 and is securely engaged with the connection piece 60 by being engaged with the ridge 611 of the connection piece 60 when the plunger 20 is inserted into the barrel 10 and the insertion 21 is inserted through the hole 622 in the connection piece 60 and into the passage 711 in the base 70. By this way, the plunger 20 is connected to the base 70 and the connection piece 60. A breaking groove 23 is defined in an outer periphery of the plunger 20 and located close to the second end of the plunger 20.

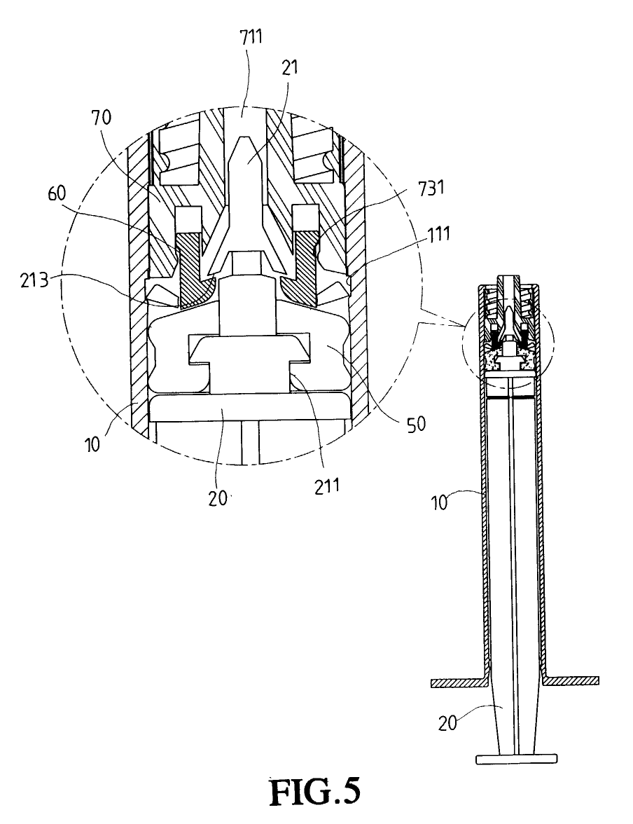

The plunger 20 can be pulled back to suck medicine into the space 11 in the barrel 10 and then be pushed toward the base 70 to inject the medicine into the patient. Referring to FIG. 5, the flange 61 of the connection piece 60 is flexible and can be easily disengaged from the first groove 111 to allow the pull of the plunger 20.

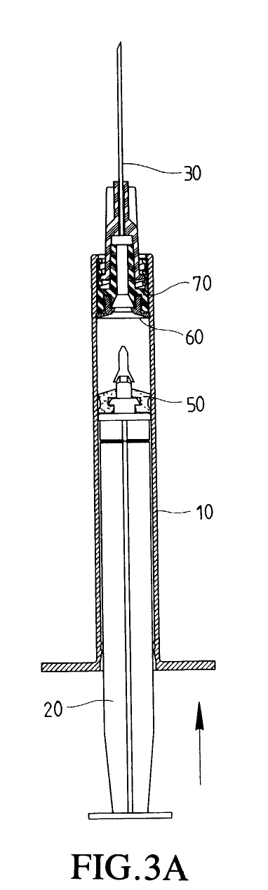

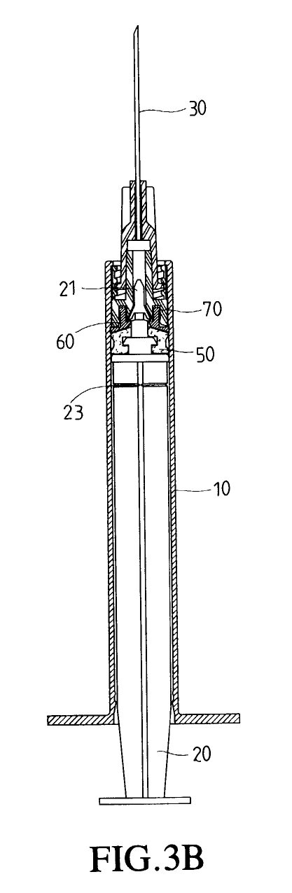

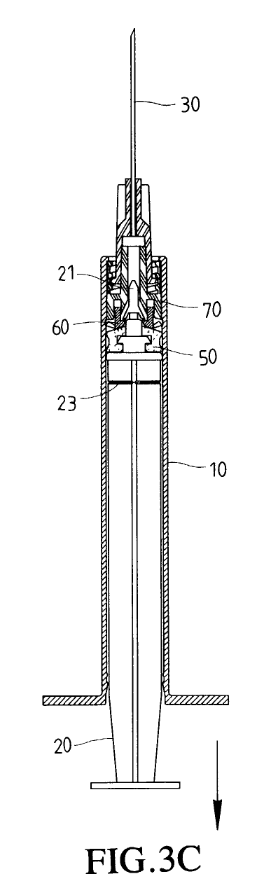

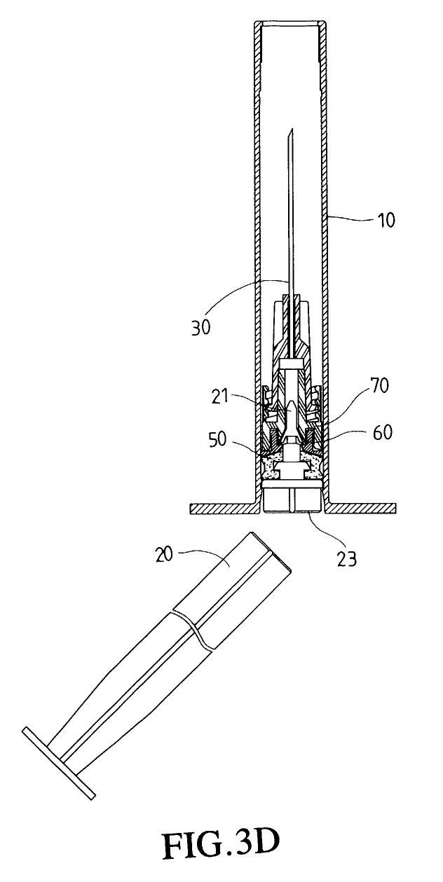

Referring to FIGS. 3C and 3D, after the infringe, the plunger 20 together with the base 70 and the needle cannula unit 30 are pulled back and the plunger 20 has a flange 22 extending from the outer periphery thereof and the flange 22 of the plunger 20 is engaged with the second groove 112 so that the user knows that the plunger 20 is positioned and the user breaks the plunger 20 at the breaking groove 23 to leave the needle cannula unit 30 in the barrel 10.

During the action, the users do not need to touch the needle cannula and the possibility of being hurt by the used needle cannula can be reduced.

While we have shown and described the embodiment in accordance with the present invention, it should be clear to those skilled in the art that further embodiments may be made without departing from the scope of the present invention.

What is claimed is:

1. A safety hypodermic syringe comprising:
a barrel having an opening defined in a first end thereof;
a base movably received in the barrel and a passage defined therethrough, an engaging recess defined in a first end of the base and a cylindrical connection piece having a hole defined therethrough and engaged with the engaging recess;
a needle cannula unit fixed connected to a second end of the base and extending out from the second end of the barrel, and
a plunger having a thumb rest on a first end thereof and an insertion extending from a second end of the plunger, the insertion inserted through the hole in the connection piece and engaged with the connection piece, the insertion inserted into the passage in the base, an enlarged skirt extending from an outer periphery of the insertion and securely engaged with the connection piece, a stopper mounted to a root portion of the insertion, a breaking groove defined in an outer periphery of the plunger and located close to the second end of the plunger.

2. The safety hypodermic syringe as claimed in claim 1, wherein the connection piece has a flange and the barrel has a first groove defined in an inner periphery thereof, the flange of the connection piece engaged with the first groove.

3. The safety hypodermic syringe as claimed in claim 1, wherein the connection piece has a ridge extending from an inner periphery of the hole of the connection piece and an enlarged skirt extending from an outer periphery of the insertion, the enlarged skirt engaged with the ridge.

4. The safety hypodermic syringe as claimed in claim 1 further comprising a flange extending from the outer periphery of the plunger and the barrel has a second groove defined in the inner periphery of the barrel, the second groove located close to the first end of the barrel, the flange of the plunger engaged with the second groove.

5. The safety hypodermic syringe as claimed in claim 1 further comprising a groove defined in the root portion of the insertion and the stopper is engaged with the groove in the root portion of the insertion.

6. The safety hypodermic syringe as claimed in claim 1, wherein the base has a threaded inner periphery and the needle cannula unit has a flange which is engaged with the threaded inner periphery of the base.

7. The safety hypodermic syringe as claimed in claim 1, wherein the base has a first connection protrusion extending from an inner periphery of the engaging recess and the connection piece has a second connection protrusion on an outer periphery thereof, the first connection protrusion engaged with the second connection protrusion.
